(19) United States
(12) Patent Application Publication
(10) Pub. No.: US 2025/0347777 A1
(43) Pub. Date: Nov. 13, 2025

(54) OBSERVATION TARGET DETECTION DEVICE AND OBSERVATION TARGET DETECTION METHOD

(71) Applicant: Murata Manufacturing Co., Ltd., Nagaokakyo-shi (JP)

(72) Inventors: Takahiro KINOSHITA, Nagaokakyo-shi (JP); Kosei ASANO, Nagaokakyo-shi (JP)

(73) Assignee: Murata Manufacturing Co., Ltd., Nagaokakyo-shi (JP)

(21) Appl. No.: 19/271,970

(22) Filed: Jul. 17, 2025

Related U.S. Application Data
(63) Continuation of application No. PCT/JP2024/002265, filed on Jan. 25, 2024.

(30) Foreign Application Priority Data
Mar. 16, 2023 (JP) 2023-042239

(51) Int. Cl. G01S 7/41; G01S 13/32; G01S 13/536; G01S 13/88

(52) U.S. Cl. CPC G01S 7/415; G01S 7/411; G01S 7/414; G01S 13/32; G01S 13/536; G01S 13/88

(57) ABSTRACT

An observation target detection device includes: a radar to identify a plurality of reflection points within an observation range based on reflected waves and circuitry to distinguish between an observation target and a non-observation target. The circuitry is configured to, for each of the plurality of reflection points, calculate a first correlation degree indicating correlation between temporal changes in a first signal property; select a set of candidate reflection points having the first correlation degree equal to or greater than a first predetermined value; calculate, when a number of reflection points selected is equal to or more than three, a second correlation degree indicating correlation between temporal changes in the first signal property for pairs of the reflection points; and determine that pairs having the second correlation degree equal to or greater than a second predetermined value are a non-observation target reflection points and otherwise are observation target reflection points.

### CROSS-REFERENCE TO RELATED APPLICATIONS

The present application is a continuation of International Application No. PCT/JP2024/002265, filed Jan. 25, 2024, which claims priority to Japanese patent application JP 2023-042239, filed Mar. 16, 2023, the entire contents of each of which being incorporated herein by reference.

### TECHNICAL FIELD

The present disclosure relates to an observation target detection device and an observation target detection method.

### BACKGROUND ART

A system has been introduced to detect a human body by using a radar (RADAR: RAdio Detection And Ranging) and acquire biometric information based on a body surface displacement of the human body. In a radar system, reflected waves of radio waves emitted from an antenna are analyzed to identify a target. It is therefore necessary to improve analysis accuracy for the reflected waves and identification accuracy for the target.

Patent Document 1 discloses an activity measurement device, an activity measurement system, an activity measurement program, and an activity measurement method for measuring a respiratory rate of a human or animal based on a median period of the interval between peaks or bottoms of time series data having the maximum amplitude of a plurality of pieces of time series data by acquiring a beat signal between signals transmitted and received by an FMCW (Frequency Modulated Continuous Wave) radar and extracting the plurality of pieces of time series data for a transmitted signal having a plurality of discrete frequencies of a sweep frequency.

Patent Document 2 discloses an observation target detection device or an observation target detection method capable of detecting a target signal even if an antenna vibrates due to wind or the like, causing changes in ground clutter or apparent Doppler frequency of a target, by correcting the Doppler velocity based on a reflected wave component from the ground surface (ground clutter) present in a received signal in a configuration using a Doppler radar to detect the target.

Patent Document 3 discloses an interference-type vibration observation device, a vibration observation program, a recording medium, a vibration observation method, and a vibration observation system for receiving reflected waves from an observation target with a receiving antenna mounted on a platform, such as a helicopter, that vibrates or fluctuates and performing vibration analysis of a fixed point determined from an image generated from observation data that represents the vibration of the observation target or a specific part to remove the vibration of the fixed point from the observation data.
Non-Patent Document 1 discloses a vital sign extraction method that utilizes a degree of correlation between the amplitude and phase of a signal obtained from a reflected wave of an FMCW radar.
Non-Patent Document 2 discloses a vital sign detection method that utilizes autocorrelation of time changes in the phase of each reflected wave and cross-correlation of Doppler components (Doppler velocities) between the reflected waves.

### CITATION LIST

Patent Document
Patent Document 1: Japanese Unexamined Patent Application Publication No. 2022-040858
Patent Document 2: Japanese Unexamined Patent Application Publication No. 2006-214766
Patent Document 3: International Publication No. 2016/027422

### Non-Patent Document

Non-Patent Document 1: H.-I. Choi, H. Song, and H.-C. Shin, “Target Range Selection of FMCW Radar for Accurate Vital Information Extraction,” IEEE Access, vol. 9, pp. 1261, 2021.
Non-Patent Document 2: E. Cardillo, C. Li, and A. Caddemi, “Vital Sign Detection and Radar Self-Motion Cancellation Through Clutter Identification,” IEEE Transactions on Microwave Theory and Techniques, vol. 69, no. 3, pp. 1932, 2021.

### SUMMARY

### Technical Problems

As for a vital sensor that acquires a body surface displacement of a stationary human body as biometric information, when the sensor itself vibrates periodically due to disturbance, there is a possibility that reflections from a stationary object (hereinafter also referred to as “clutter”) in the environment in which the biometric information is acquired may be erroneously determined as reflections from the human body. There is also a possibility that the periodic displacements caused by the vibration of the sensor are superimposed, making acquisition of highly accurate biometric information impossible.

In the technology described in Patent Document 1, the respiratory rate is acquired based on the median period of the interval between the peaks or bottoms of the time series data having the maximum amplitude. Therefore, when the periodic vibration of the sensor itself is relatively large compared to the body surface displacement of the human body, discrimination between reflections from the human body and the clutter may not be possible. Suppression of the influence of the periodic vibration of the sensor itself is also difficult.

In the technology described in Patent Document 2, target information is detected by correcting the Doppler velocity based on the ground clutter, which is the reflected wave from the stationary ground surface, relative to changes in apparent Doppler frequency of the received signal due to antenna vibration or the like in a radar device for detecting a ground target. Thus, applying this technology to a vital sensor that acquires minute body surface displacements of a stationary human body as biometric information is difficult.

In the technology described in Patent Document 3, a fixed point (stationary point) needs to be determined from the image generated from the observation data that represents the vibration of the observation target or a specific part. For this reason, this technology cannot be applied to a situation where reflections from the human body cannot be discriminated from clutter in the environment in which the biometric information is acquired.

In the technology described in Non-Patent Document 1, a range bin is selected, which has a high correlation between amplitude and phase. In Non-Patent Document 1, when each range bin contains a vibration component of the sensor, a range bin including no vital signs, such as breathing or heart rate, may also have high correlation between amplitude and phase, making discrimination between reflections from the human body and the clutter impossible.

In the technology described in Non-Patent Document 2, a signal with high autocorrelation of time changes in the phase, that is, a signal whose phase changes periodically, is determined as a reflection from the human body. When the cross-correlation of the Doppler components between relative signals is high, the correlation due to radar self-motion effects (RSMs) is high and that the signal is clutter. Therefore, when the RSMs have periodicity, the possibility for the clutter to be erroneously determined as the reflection from the human body or for the reflection from the human body to be erroneously determined as the clutter is increased, making discrimination between the reflection from the human body and the clutter difficult.

The present disclosure has been made in view of the above, and is directed to realizing an observation target detection device and an observation target detection method capable of appropriately discriminating between an observation target and a non-observation target.

### Solutions to Problems

An observation target detection device according to an aspect of the present disclosure includes: a radar that emits radio waves into an observation range to specify, based on reflected waves of the radio waves, a position of a reflection point within the observation range; a first correlation degree calculation unit that calculates a first correlation degree indicating a level of correlation between temporal changes in at least one of amplitude, intensity, and power of a signal at the reflection point and temporal changes in phase; a first determination unit that selects a reflection point at which the first correlation degree is equal to or greater than a predetermined value; a second correlation degree calculation unit that calculates, when a number of reflection points selected by the first determination unit is equal to or more than three, a second correlation degree indicating a level of correlation between temporal changes in at least one of the amplitude, intensity, power, and phase between signals at two of the reflection points; and a second determination unit that determines that one of the reflection points at which the second correlation degree is equal to or greater than a predetermined value is a non-observation target reflection point and that one of the reflection points other than the non-observation target reflection point is an observation target reflection point.

In this configuration, for each reflection point specified by the radar, a reflection point including a periodic fluctuation component that is relatively large compared to random noise components is selected by calculating the first correlation degree indicating the level of correlation between temporal changes in at least one of the amplitude, intensity, and power of the signal at each reflection point and temporal changes in phase and selecting a reflection point at which the calculated first correlation degree is equal to or greater than a predetermined threshold. Then, the second correlation degree indicating the level of correlation between temporal changes in at least one of the amplitude, intensity, power, and phase between the signals of the selected reflection points is calculated. Each reflection point at which the second correlation degree is equal to or greater than a predetermined threshold is determined as a non-observation target reflection point, and reflection points other than the non-observation target reflection point are determined as observation target reflection points. This makes it possible to appropriately discriminate between the observation target reflection point and the non-observation target reflection points including no displacement at the observation target reflection point.

An observation target detection method according to another aspect of the present disclosure includes: a first step of emitting radio waves into an observation range of a radar to specify, based on reflected waves of the radio waves, a position of a reflection point within the observation range; a second step of calculating a first correlation degree indicating a level of correlation between temporal changes in at least one of amplitude, intensity, and power of a signal at the reflection point and temporal changes in phase; a third step of selecting a reflection point at which the first correlation degree is equal to or greater than a predetermined value; a fourth step of calculating, when a number of reflection points selected in the third step is equal to or more than three, a second correlation degree indicating a level of correlation between temporal changes in at least one of the amplitude, intensity, power, and phase between signals at two of the reflection points; and a fifth step of determining that one of the reflection points at which the second correlation degree is equal to or greater than a predetermined value is a non-observation target reflection point and that one of the reflection points other than the non-observation target reflection point is an observation target reflection point.

According to this configuration, the position of the reflection point within the observation range 2 of the radar is specified in position specification processing (first step), and first correlation degree calculation processing (second step) is executed on each reflection point specified in the position specification processing to calculate the first correlation degree indicating the level of correlation between temporal changes in at least one of the amplitude, intensity, and power of the signal at each reflection point and temporal changes in phase. Then, first determination processing (third step) is executed to select a reflection point at which the first correlation degree is equal to or greater than a predetermined threshold, thereby selecting a reflection point including a periodic fluctuation component that is relatively large compared to a random noise component. Thereafter, second correlation degree calculation processing (fourth step) is executed to calculate a second correlation degree indicating the level of correlation between temporal changes in amplitude, intensity, power, and phase between the signals at the reflection points selected in the first determination processing. Then, second determination processing (fifth step) is executed to determine that one of the reflection points at which the second correlation degree calculated in the second correlation degree calculation processing is equal to or greater than a predetermined threshold is a non-observation target reflection point, and that one of the reflection points other than the non-observation target reflection point is an observation target reflection point. This makes it possible to appropriately discriminate between the observation target reflection point and the non-observation target reflection point including no displacement at the observation target reflection point.

### Advantageous Effects

The present disclosure can realize an observation target detection device and an observation target detection method capable of appropriately discriminating between an observation target and a non-observation target.

### BRIEF DESCRIPTION OF DRAWINGS

FIG. 1 is a block diagram showing a schematic configuration of an observation target detection device according to an embodiment.

FIG. 2 is a conceptual diagram showing the positions of reflection points within an observation range of the observation target detection device according to the embodiment.

FIG. 3A is a first diagram showing an example of temporal changes in signal amplitude at a reflection point.

FIG. 3B is a first diagram showing an example of temporal changes in signal phase at the reflection point.

FIG. 4A is a second diagram showing an example of temporal changes in signal amplitude at the reflection point.

FIG. 4B is a second diagram showing an example of temporal changes in signal phase at the reflection point.

FIG. 5A is a first diagram showing an example of temporal changes in signal phase when the reflection point is a human body.

FIG. 5B is a second diagram showing an example of temporal changes in signal phase when the reflection point is the human body.

FIG. 5C is a diagram showing an example of temporal changes in signal phase when the reflection point is a stationary object.

FIG. 6A is a first diagram showing an example of temporal changes in signal amplitude when the reflection point is a human body.

FIG. 6B is a second diagram showing an example of temporal changes in signal amplitude when the reflection point is a human body.

FIG. 6C is a first diagram showing an example of temporal changes in signal amplitude when the reflection point is a stationary object.

FIG. 6D is a second diagram showing an example of temporal changes in signal amplitude when the reflection point is a stationary object.

FIG. 7 is a flowchart showing an example of observation target detection processing by the observation target detection device according to the embodiment.

FIG. 8 is a flowchart showing an example of first correlation degree calculation processing (second step).

FIG. 9 is a flowchart showing an example of first determination processing (third step).

FIG. 10 is a flowchart showing an example of second correlation degree calculation processing (fourth step).

FIG. 11 is a flowchart showing an example of second determination processing (fifth step).

FIG. 12 is a flowchart showing an example of displacement calculation processing (sixth step).

FIG. 13 is a sub-flowchart showing an example of first processing of the displacement calculation processing (sixth step).

FIG. 14 is a sub-flowchart showing an example of second processing of the displacement calculation processing (sixth step).

FIG. 15 is a flowchart showing an example of disturbance removal processing (seventh step).

### DESCRIPTION OF EMBODIMENTS

An observation target detection device and an observation target detection method according to an embodiment will be described in detail below with reference to the drawings. Note that the present disclosure is not limited to this embodiment.

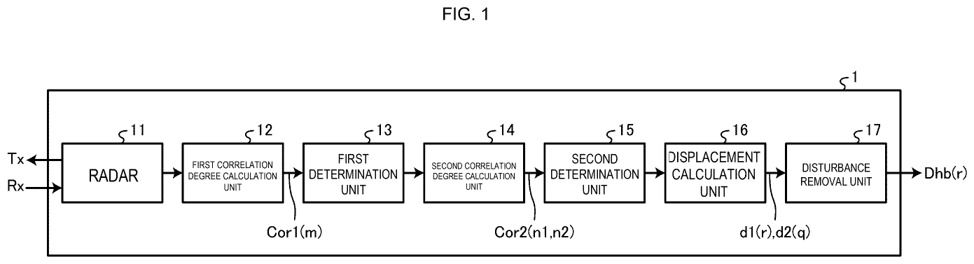

FIG. 1 is a block diagram showing a schematic configuration of the observation target detection device according to the embodiment. An observation target detection device 1 according to the embodiment includes a radar 11, a first correlation degree calculation unit 12, a first determination unit 13, a second correlation degree calculation unit 14, a second determination unit 15, a displacement calculation unit 16, and a disturbance removal unit 17. As used herein, “unit” refers to circuitry that may be configured via the execution of computer readable instructions, and the circuitry may include one or more local processors (e.g., CPU's), and/or one or more remote processors, such as a cloud computing resource, or any combination thereof.

The radar 11 emits radio waves (transmission waves Tx) of, for example, millimeter wave bands or microwave bands into an observation range in which an observation target is to be detected by the observation target detection device according to the embodiment, and receives radio waves (reflected waves Rx) reflected at an unknown reflection point to identify the position of the reflection point within the observation range. Examples of the radar 11 include an FMCW (Frequency Modulated Continuous Wave) radar, a Doppler radar, a pulse radar, and the like. In the present disclosure, the radar 11 may be configured to measure at least the distance and angle (orientation) to a reflection point.

The radar 11 performs AD conversion processing, filtering processing, various FFT processing, and the like on the received reflected wave Rx to identify the position of the reflection point within the observation range. The present disclosure is not limited to the specific processing in the radar 11.

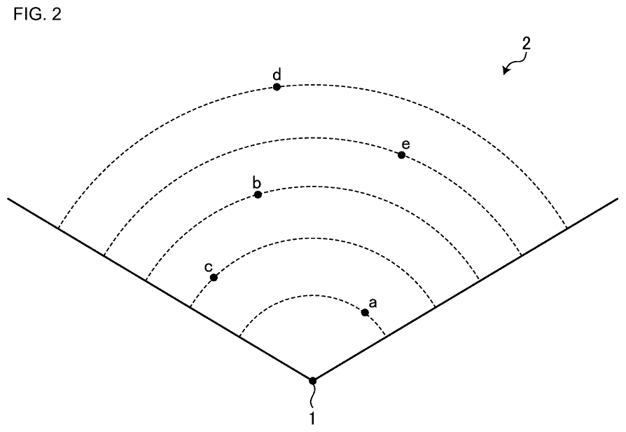

FIG. 2 is a conceptual diagram showing the positions of reflection points within the observation range of the observation target detection device according to the embodiment. In FIG. 2, a, b, c, d, and e represent reflection points identified by the radar 11 in an observation range 2 of the observation target detection device 1.

In the present disclosure, the observation target detection device 1 may be a vital sensor that acquires, for example, a body surface displacement of a human body as biometric information and detects vital signs such as heart rate, heart rate variability, respiratory rate, and respiratory depth. In this case, the reflection points a, b, c, d, and e to be identified within the observation range 2 of the observation target detection device 1 include not only the human body that is the observation target of the observation target detection device 1, but also stationary objects present in the biometric information acquisition environment.

When the biometric information acquisition environment is a mobile object such as an automobile, the stationary object present in the vital sign acquisition environment may be a seat, a dashboard, or the like. Alternatively, when the biometric information acquisition environment is a hospital examination room, a patient room, or the like, the stationary object may be a wall, a bed, or the like. In order to improve the detection accuracy of the vital signs, it is necessary to appropriately determine whether or not the reflection point identified by the radar 11 is a human body.

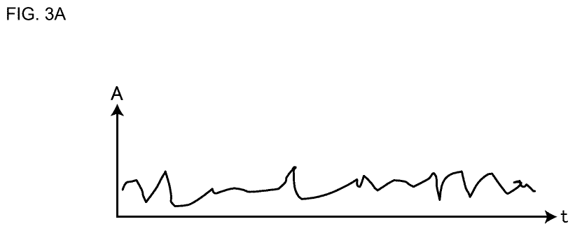

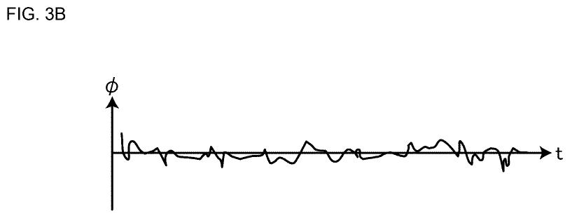

FIG. 3A is a first diagram showing an example of temporal changes in signal amplitude at the reflection point. FIG. 3B is a first diagram showing an example of temporal changes in signal phase at the reflection point. FIGS. 3A and 3B each show an example where the stationary object within the observation range 2 is identified as the reflection point.

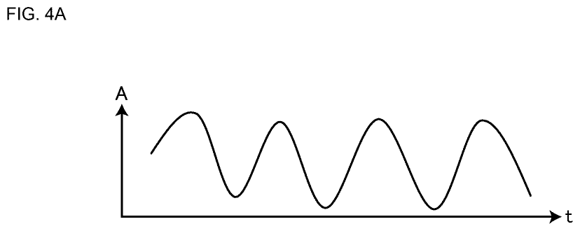

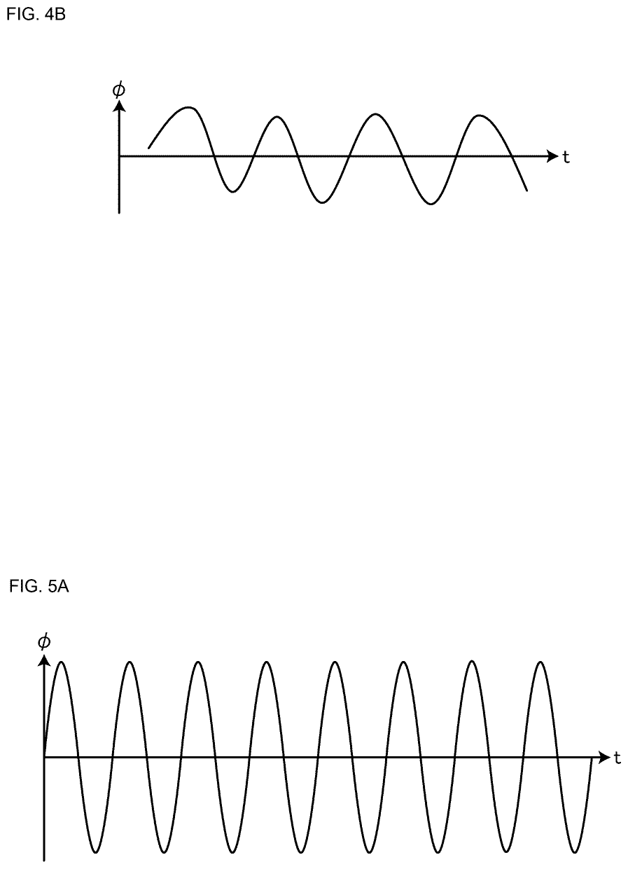

FIG. 4A is a second diagram showing an example of temporal changes in signal amplitude at the reflection point. FIG. 4B is a second diagram showing an example of temporal changes in signal phase at the reflection point. FIGS. 4A and 4B each show an example where a stationary human body is identified as the reflection point.

Noise components are superimposed on a signal corresponding to each reflection point. Therefore, even if the reflection point is the stationary object, the noise components appear as temporal changes in amplitude and phase of the signal, as shown in FIGS. 3A and 3B. Such noise components are so-called random noise, and there is no correlation between the noise components appearing in the temporal changes in amplitude and the noise components appearing in the temporal changes in phase.

When the reflection point is the stationary human body, on the other hand, substantially periodic body surface displacements of the human body such as breathing and heartbeat are superimposed, in addition to the random noise components. These body surface displacements are relatively large compared to the random noise components and are dominant over the temporal changes in amplitude and phase. Therefore, when the reflection point is the stationary human body, the correlation between the temporal changes in amplitude and the temporal changes in phase is high, as shown in FIGS. 4A and 4B.

In the present disclosure, a reflection point with a high degree of correlation between temporal changes in amplitude and temporal changes in phase of the signal is selected from among the reflection points identified by the radar 11. Thus, reflection points including no body surface displacements of the human body may be excluded.

Specifically, the first correlation degree calculation unit 12 uses Formula (1) below to calculate a first correlation degree cor1(m), which indicates the level of correlation between temporal changes in amplitude A(m) and temporal changes in phase φ(m) of the signal at each of the reflection points m (reflection points a, b, c, d, and e in the example shown in FIG. 2. In this case, the total number M of the reflection points m is 5).

$$\begin{matrix}[ Math 1 ] & \\ Cor 1 ( m ) = \frac{{\int }_{0}^{T} {A}_{m} ( t ) {ϕ}_{m} ( t ) dt}{{σ}_{{A}_{m}} {σ}_{{ϕ}_{m}}} & ( 1 )\end{matrix}$$

In Formula (1) above, σA(m) represents the standard deviation of the amplitude A(m), and oφ(m) represents the standard deviation of the phase φ(m). The normalized first correlation degree cor1(m) is thus obtained.

When the first correlation degree cor1(m) calculated by the first correlation degree calculation unit 12 is equal to or greater than a predetermined threshold Cor1th (for example, 0.9) (Cor1(m)≥Cor1th), the first determination unit 13 selects any of the reflection points m (the reflection points a, b, c, d, and e in the example shown in FIG. 2) as a reflection point n (the total number of selected reflection points n is N). As a result, at least the reflection points including no body surface displacement of the human body are not selected. The threshold Cor1th for the first correlation degree cor1(m) may be, for example, about Cor1th=0.99 when the ratio of the noise component to the signal is equal to or less than 1/100.

Note that Formula (1) above shows the example of calculating the first correlation degree cor1(m) between the temporal change in amplitude A(m) and the temporal change in phase φ(m) of the signal at each of the reflection points m. However, instead of the amplitude A, the first correlation degree may be calculated using the intensity or power of a signal on which the body surface displacement of the human body is superimposed.

Here, in a case where the observation target detection device 1 itself according to the embodiment vibrates periodically, the periodic vibration of the observation target detection device 1 is superimposed on a signal corresponding to each reflection point.

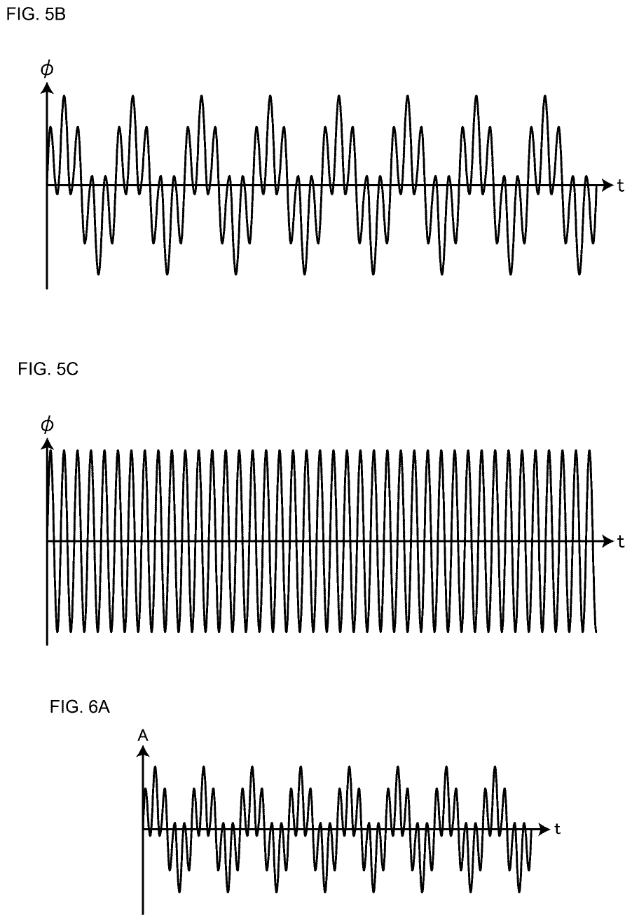

FIG. 5A is a first diagram showing an example of temporal changes in signal phase when the reflection point is a human body. FIG. 5B is a second diagram showing an example of temporal changes in signal phase when the reflection point is the human body. FIG. 5C is a diagram showing an example of temporal changes in signal phase when a reflection point is a stationary object.

FIGS. 5A, 5B, and 5C each show an example of a simulation in which the vibration of the observation target detection device 1 is a sine wave of 1 Hz, and the body surface displacement of the human body is a sine wave of 0.2 Hz. Note that while FIGS. 5A, 5B, and 5C each show the example of temporal changes in signal phase at each reflection point, but the same applies to temporal changes in signal amplitude at each reflection point.

When the reflection point is the human body and the observation target detection device 1 is not vibrating, a sine wave of 0.2 Hz simulating the body surface displacement is superimposed on the temporal changes in signal phase (or amplitude) at the reflection point, as shown in FIG. 5A. When the observation target detection device 1 is vibrating at 1 Hz, on the other hand, a sine wave of 1 Hz simulating the vibration of the observation target detection device 1 is superimposed on the temporal changes in signal phase (or amplitude) at the reflection point, in addition to the sine wave of 0.2 Hz simulating the body surface displacement, as shown in FIG. 5B.

When the periodic vibration of the observation target detection device 1 is relatively large compared to the random noise components, even if the reflection point is a stationary object, the periodic vibration of the observation target detection device 1 becomes dominant over the temporal changes in phase (and amplitude), as shown in FIG. 5C. This increases the correlation between the temporal changes in amplitude and the temporal changes in phase, which may cause the first correlation degree calculated by the first correlation degree calculation unit 12 to be equal to or greater than the threshold value, leading to selection by the first determination unit 13.

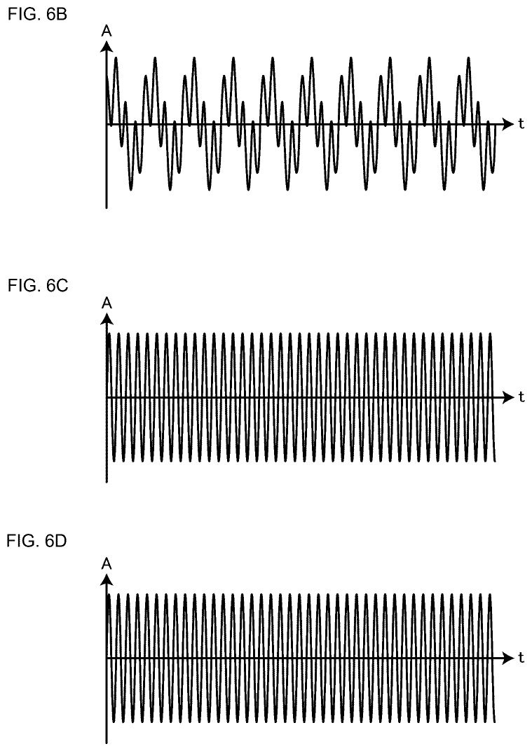

FIG. 6A is a first diagram showing an example of temporal changes in signal amplitude when the reflection point is a human body. FIG. 6B is a second diagram showing an example of temporal changes in signal amplitude when the reflection point is a human body. FIG. 6C is a first diagram showing an example of temporal changes in signal amplitude when the reflection point is a stationary object. FIG. 6D is a second diagram showing an example of temporal changes in signal amplitude when the reflection point is a stationary object.

In FIGS. 6A, 6B, 6C, and 6D, the vibration of the observation target detection device 1 is a sine wave of 1 Hz. FIG. 6A shows an example of a simulation in which the body surface displacement of a human body A is a sine wave of 0.2 Hz. FIG. 6B shows an example of a simulation in which the body surface displacement of human body B is a sine wave of 0.25 Hz. FIG. 6C shows an example of simulating a stationary object C. FIG. 6D shows an example of simulating a stationary object D. Note that FIGS. 6A, 6B, 6C, and 6D each show the example of temporal changes in signal amplitude at each reflection point, but the same applies to temporal changes in signal phase at each reflection point.

The correlation of temporal changes in amplitude (or phase) is low between the signals of the human body (human body A or human body B) and the stationary object (stationary object C or stationary object D), and is high between the signals of the stationary objects (between the stationary object C and the stationary object D). On the other hand, the frequency of the body surface displacement differs between the human bodies (between the human body A and the human body B) (the frequency of the body surface displacement of the human body A is 0.2 Hz, and the frequency of the body surface displacement of the human body B is 0.25 Hz). This reduces the correlation of temporal changes in amplitude (or phase) between the signals.

Therefore, in the present disclosure, any pair of reflection points selected by the first determination unit 13 that have a high degree of correlation between temporal changes in their respective signals (e.g., amplitude or phase) are determined to be stationary objects, which are classified as non-observation targets. Thus, the reflection point other than the reflection point determined as the stationary object is determined as the human body that is the observation target of the observation target detection device 1.

Specifically, the second correlation degree calculation unit 14 uses Formula (2) below to calculate a second correlation degree cor2(n1, n2), which indicates the level of correlation between temporal changes in amplitude A(n1) of a signal at a reflection point n1 (n1 is an integer from 1 to N) selected by the first determination unit 13 and temporal changes in amplitude A(n2) of a signal at a reflection point n2 (n2 is an integer from 1 to N, excluding n1).

$$\begin{matrix}[ Math 2 ] & \\ Cor 1 ( n 1 , n 2 ) = \frac{{\int }_{0}^{T} {A}_{n 1} ( t ) {A}_{n 2} ( t ) dt}{{σ}_{{A}_{n 1}} {σ}_{{A}_{n 2}}} & ( 2 )\end{matrix}$$

In Formula (2) above, σA(n1) represents the standard deviation of the amplitude A(n1) of the signal corresponding to the reflection point n1, and σA(n2) represents the standard deviation of the amplitude A(n2) of the signal corresponding to the reflection point n2. The normalized second correlation degree cor2(n1, n2) is thus obtained.

Alternatively, the second correlation degree calculation unit 14 uses Formula (3) below to calculate a second correlation degree cor2(n1, n2), which indicates the level of correlation between temporal changes in phase φ(n1) of the signal at the reflection point n1 (n1 is an integer from 1 to N) selected by the first determination unit 13 and temporal changes in phase q (n2) of the signal at the reflection point n2 (n2 is an integer from 1 to N, excluding n1).

$$\begin{matrix}[ Math 3 ] & \\ Cor 2 ( n 1 , n 2 ) = \frac{{\int }_{0}^{T} {ϕ}_{n 1} ( t ) {ϕ}_{n 2} ( t ) dt}{{σ}_{{ϕ}_{n 1}} {σ}_{{ϕ}_{n 2}}} & ( 3 )\end{matrix}$$

In Formula (3) above, σφ(n1) represents the standard deviation of the phase φ(n1) of the signal corresponding to the reflection point n1, and φ(n2) represents the standard deviation of the phase φ(n2) of the signal corresponding to the reflection point n2. The normalized second correlation degree cor2(n1, n2) is thus obtained.

When the second correlation degree cor2(n1, n2) calculated by the second correlation degree calculation unit 14 is equal to or greater than a predetermined threshold Cor2th (for example, 0.9) (Cor2 (n1, n2)≥Cor2th), the second determination unit 15 determines that the reflection points n1 and n2 are non-observation target reflection points q. After threshold determination of all the second correlation degrees calculated by the second correlation degree calculation unit 14, the reflection points excluding the non-observation target reflection points q are determined to be observation target reflection points r (if the total number of the non-observation target reflection points q is Q, the total number of the observation target reflection points r is R (R=N-Q)). As with the threshold Cor1th for the first correlation degree cor1(m), the threshold Cor2th for the second correlation degree cor2(n1, n2) may be about Cor2th=0.99.

Formula (2) above shows the example where the second correlation degree cor2(n1, n2) between the temporal changes in amplitude A(n1) of the signal at the reflection point n1 and the temporal changes in amplitude A(n2) of the signal at the reflection point n2 is calculated. Formula (3) above shows the example where the second correlation degree cor2(n1, n2) between the temporal changes in phase φ(n1) of the signal at the reflection point n1 and the temporal changes in phase φ(n2) of the signal at the reflection point n2 is calculated. However, instead of the amplitude A or the phase φ, the second correlation degree may be calculated using the intensity or power of a signal on which the body surface displacement of the human body and the periodic vibration of the observation target detection device 1 are superimposed. Alternatively, when the radar 11 is the FMCW radar, the second correlation degree may be calculated using a complex signal corresponding to each reflection point.

The displacement calculation unit 16 calculates the displacements of all the observation target reflection points r and non-observation target reflection points q (observation target reflection point displacement d1(r) and non-observation target reflection point displacement d2(q)).

The displacement d of each reflection point can be calculated from the phase φ of the signal at each reflection point. The phase φ of the signal at each reflection point is calculated by Formula (4) below, where λ is the center frequency of the radio waves (transmitted wave Tx and reflected wave Rx) used by the radar 11.

$$φ = 4 π \times d / λ \qquad ( 4 )$$

As described above, when the observation target detection device 1 itself according to the embodiment vibrates periodically, the periodic vibration of the observation target detection device 1 is superimposed on the phase of the signal corresponding to each reflection point. Therefore, to obtain the body surface displacement of the human body determined to be the observation target reflection point r by the second determination unit 15, displacement components caused by the vibration of the observation target detection device 1 need to be removed from the displacement at the observation target reflection point r.

Here, when the periodic vibration of the observation target detection device 1 is relatively large compared to the random noise components, even if the reflection point is a stationary object, the periodic vibration of the observation target detection device 1 becomes dominant over the temporal changes in phase as described above (see FIG. 5C). Therefore, as for the displacement calculated from the phase of the stationary object determined to be the non-observation target reflection point q by the second determination unit 15, the displacement component caused by the periodic vibration of the observation target detection device 1 becomes dominant.

Therefore, in the present disclosure, the displacement component of the stationary object is removed from the displacement at the observation target reflection point r. Thus, highly accurate biometric information (a body surface displacement of the human body) may be acquired in which displacement components caused by the periodic vibration of the observation target detection device 1 are suppressed, and the detection accuracy of vital signs may be improved.

Specifically, the disturbance removal unit 17 extracts, from among the reflection points determined as the non-observation target reflection points q by the second determination unit 15, the displacement corresponding to the non-observation target reflection point with the maximum signal amplitude as a non-observation target reflection point displacement d2max, and calculates a body surface displacement Dhb(r) of the human body by removing a displacement component Δd(˜d2max) proportional to the non-observation target reflection point displacement d2max from the observation target reflection point displacement d1 (r) calculated by the displacement calculation unit 16 (Dhb(r)=d1(r)−Δd). In the present disclosure, description is given of the example where, among the reflection points determined as the non-observation target reflection points q by the second determination unit 15, the displacement corresponding to the non-observation target reflection point with the maximum signal amplitude is extracted as the non-observation target reflection point displacement d2max. However, instead of the amplitude, the displacement corresponding to the non-observation target reflection point with the maximum signal intensity or power may be extracted as the non-observation target reflection point displacement d2max.

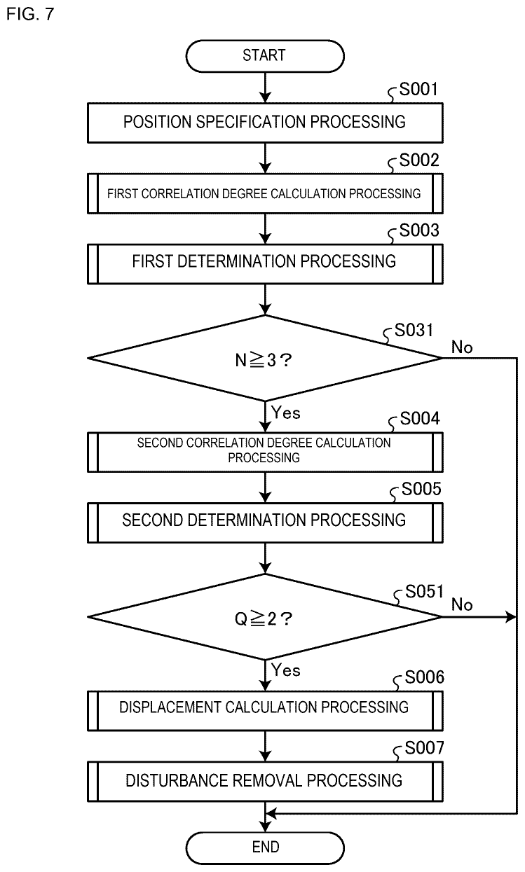

A specific example of observation target detection processing in the observation target detection device 1 according to the embodiment will be described below. FIG. 7 is a flowchart showing an example of the observation target detection processing by the observation target detection device according to the embodiment.

The observation target detection device 1 according to the embodiment first executes position specification processing of a reflection point in the observation range 2 (Step S001, first step). Specifically, the radar 11 emits a transmission wave Tx into the observation range 2 and receives a reflected wave Rx reflected at an unknown reflection point to specify the position of the reflection point (for example, each of the reflection points a, b, c, d, and e shown in FIG. 2) in the observation range 2.

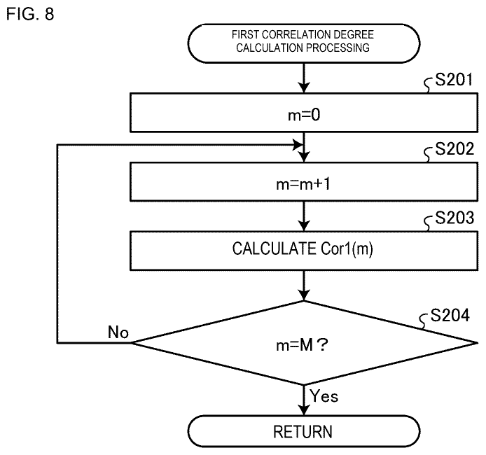

Back to the observation target detection processing shown in FIG. 7, the observation target detection device 1 then executes first correlation degree calculation processing (Step S002, second step) to calculate a first correlation degree indicating the level of correlation between temporal changes in amplitude and temporal changes in phase of the signal at each of the reflection points m by numbering each reflection point specified in the position specification processing (Step S001, first step) by the radar 11 with a number m from 1 to M (M is the total number of the specified reflection points). FIG. 8 is a flowchart showing an example of the first correlation degree calculation processing (second step S002).

In the first correlation degree calculation processing (second step S002), the first correlation degree calculation unit 12 first initializes the number m of the reflection point specified by the radar 11. Specifically, the first correlation degree calculation unit 12 sets the number m to “0” (m=0, Step S201). The first correlation degree calculation unit 12 then increments the number m (m=m+1, Step S202) and uses Formula (1) above to calculate a first correlation degree cor1(m) (Step S203).

After calculating the first correlation degree cor1(m) (Step S203), the first correlation degree calculation unit 12 determines whether or not the number m is M (Step S204). When the number m is not M (Step S204; No), in other words, when there is a reflection point for which the first correlation degree calculation processing has not been performed, the processing is repeated from Step S202. When the number m is M (m=M, Step S204; Yes), in other words, when the first correlation degree calculation processing is executed for all the reflection points specified by the radar 11, the processing returns to the observation target detection processing shown in FIG. 7. Through the first correlation degree calculation processing (second step S002), the first correlation degrees are calculated for all the reflection points specified by the radar 11.

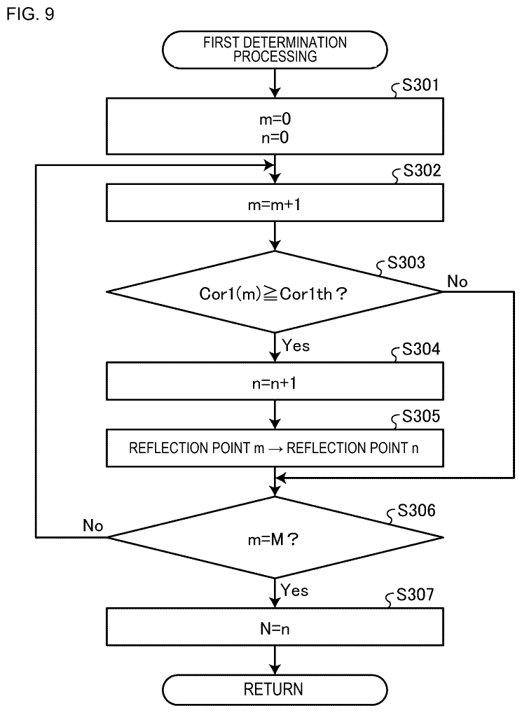

Back to the observation target detection processing shown in FIG. 7, the observation target detection device 1 then executes first determination processing (Step S003, third step) to select a reflection point at which the first correlation degree calculated by the first correlation degree calculation unit 12 is equal to or greater than a predetermined threshold. FIG. 9 is a flowchart showing an example of the first determination processing (third step S003).

In the first determination processing (third step S003), the first determination unit 13 first initializes the number m of the reflection point specified by the radar 11, and the number n up to the total number N (unknown) of reflection points selected in the first determination processing (third step S003). Specifically, the first determination unit 13 sets the number m and the number n to “0” (m=0, n=0, Step S301). The first determination unit 13 then increments the number m (m=m+1, Step S302) and determines whether or not the first correlation degree cor1(m) calculated by the first correlation degree calculation unit 12 is equal to or greater than a predetermined threshold Cor1th (Cor1(m)≥Cor1th, Step S303). When the first correlation degree cor1(m) is less than the threshold Cor1th (Cor1(m)<Cor1th, Step S303; No), the processing moves to Step S306.

When the first correlation degree cor1(m) is equal to or greater than the threshold Cor1th (Cor1(m)≥Cor1th, Step S303; Yes), the first determination unit 13 increments the number n (n=n+1, Step S304) and selects the reflection point m as the reflection point n (Step S305) before moving on to Step S306.

The processing then moves to Step S306, where the first determination unit 13 determines whether or not the number m is M (Step S306).

When the number m is not M (Step S306; No), in other words, when there is a reflection point for which the threshold determination processing of the first correlation degree has not been performed, the processing is repeated from Step S302. When the number m is M (m=M, Step S306; Yes), in other words, when the threshold determination processing of the first correlation degree is completed for all the reflection points specified by the radar 11, the number n is set to the total number N of the reflection points selected in the first determination processing (third step S003) (N=n, Step S307). Then, the processing returns to the observation target detection processing shown in FIG. 7. Through the first determination processing (third step S003), the threshold determination processing of the first correlation degree is executed for all the reflection points specified by the radar 11, and the reflection point n at which the first correlation degree is equal to or greater than the threshold Cor1th is selected.

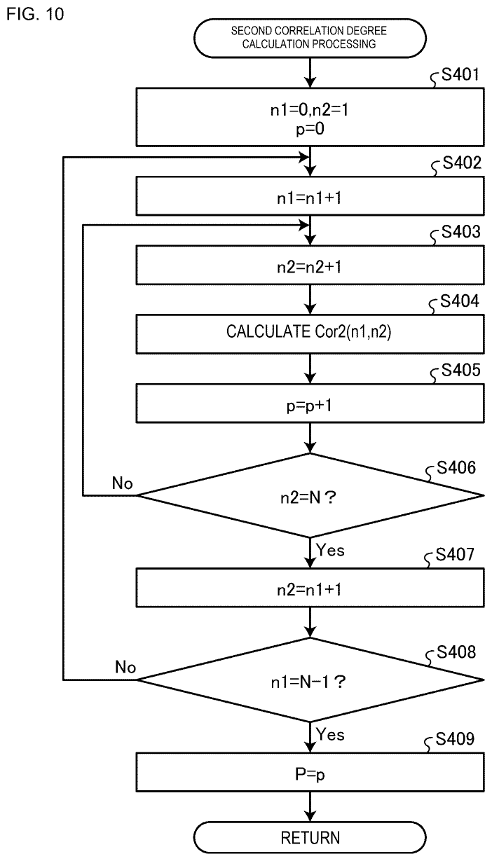

Back to the observation target detection processing shown in FIG. 7, the observation target detection device 1 determines whether or not the total number N of the reflection points n selected in the first determination processing (third step S003) is equal to or more than 3 (N23, Step S031). When N≥3 (Step S031; Yes), the observation target detection device 1 then executes second correlation degree calculation processing (Step S004, fourth step) to calculate a second correlation degree indicating the level of correlation between temporal changes in amplitude (or phase) between the signals at the reflection points selected by the first determination unit 13. FIG. 10 is a flowchart showing an example of the second correlation degree calculation processing (fourth step S004).

In the second correlation degree calculation processing (fourth step S004), the second correlation degree calculation unit 14 first initializes the number n1 (n1 is an integer from 1 to N) and the number n2 (n2 is an integer from 1 to N, excluding n1) corresponding to the number n of the reflection point selected in the first determination processing (third step S003), and also initializes the number p up to the total number P (=N×(N−1)/2) of second correlation degrees calculated in the second correlation degree calculation processing (fourth step S004). Specifically, the second correlation degree calculation unit 14 sets the number n1 to “0”, the number n2 to “1”, and the number p to “0” (n1=0, n2=1, p=0, Step S401). Next, the second correlation degree calculation unit 14 increments the number n1(n1=n1+1, Step S402) and also increments the number n2 (n2=n2+1, Step S403). The second correlation degree calculation unit 14 then uses Formula (2) or Formula (3) above to calculate the second correlation degree cor2(n1, n2) (Step S404).

After calculating the second correlation degree cor2(n1, n2) (Step S404), the second correlation degree calculation unit 14 increments the number p (p=p+1, Step S405) and determines whether or not the number n2 is N (Step S406). When the number n2 is not N (Step S406; No), the processing is repeated from Step S403. When the number n2 is N (Step S406; Yes), the second correlation degree calculation unit 14 sets the value obtained by incrementing the number n1 to the number n2 (Step S407).

Then, the second correlation degree calculation unit 14 determines whether or not the number n1 is N−1 (Step S408). When the number n1 is not N−1 (Step S408; No), the processing is repeated from Step S402. When the number n1 is N−1 (Step S408; Yes), the number p is set to the total number P of the second correlation degrees calculated in the second correlation degree calculation processing (fourth step S004) (P=p, Step S409), and then the processing returns to the observation target detection processing shown in FIG. 7. The second correlation degree calculation processing (fourth step S004) calculates the second correlation degrees between the signals at all the reflection points selected by the first determination unit 13.

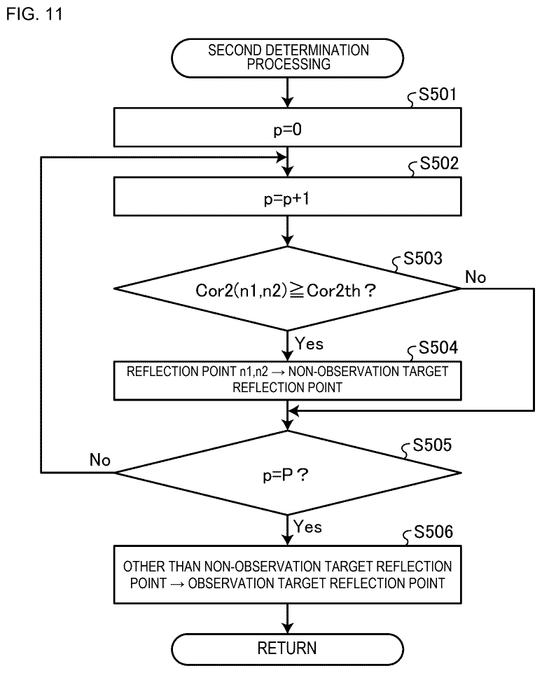

Back to the observation target detection processing shown in FIG. 7, the observation target detection device 1 then executes second determination processing (Step S005, fifth step) to determine that each of the reflection points at which the second correlation degree calculated by the second correlation degree calculation unit 14 is equal to or greater than a predetermined threshold is a non-observation target reflection point, and that reflection points other than the non-observation target reflection point are observation target reflection points. FIG. 11 is a flowchart showing an example of the second determination processing (fifth step S005).

In the second determination processing (fifth step S005), the second determination unit 15 first initializes the number p of the second correlation degree calculated by the second correlation degree calculation unit 14. Specifically, the second determination unit 15 sets the number p to “0” (p=0, Step S501). The second determination unit 15 then increments the number p (p=p+1, Step S502) and determines whether or not the second correlation degree cor2(n1, n2) calculated by the second correlation degree calculation unit 14 is equal to or greater than a predetermined threshold Cor2th (Cor2 (n1, n2)≥Cor2th, Step S503). When the second correlation degree cor2(n1, n2) is less than the threshold Cor2th (Cor2 (n1, n2)<Cor2th, Step S503; No), the processing moves to Step S505.

When the second correlation degree cor2(n1, n2) is equal to or greater than the threshold Cor2th (Cor2(n1, n2)≥Cor2th, Step S503; Yes), the reflection point n1 and the reflection point n2 are determined to be non-observation target reflection points (Step S504), and then the processing moves to Step S505.

When the processing moves to Step S505, the second determination unit 15 determines whether or not the number p is P (p=P, Step S505). When the number p is not P (Step S505; No), the processing is repeated from Step S502. When the number p is P (Step S505; Yes), the second determination unit 15 determines that the reflection point not determined to be the non-observation target reflection point in Step S504 is the observation target reflection point (Step S506). Then, the processing returns to the observation target detection processing shown in FIG. 7. Through the second determination processing (fifth step S005), all the reflection points selected by the first determination unit 13 are determined to be either the non-observation target reflection points or the observation target reflection points.

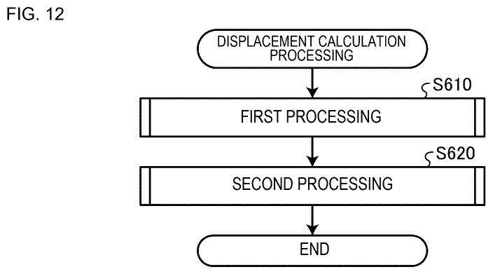

Back to the observation target detection processing shown in FIG. 7, the observation target detection device 1 assigns the number q from 1 to Q (Q is the total number of reflection points determined to be non-observation target reflection points) to each reflection point determined to be the non-observation target reflection point in the second determination processing (Step S005, fifth step) by the second determination unit 15, and similarly assigns the number r from 1 to R (R is the total number of reflection points determined to be the observation target reflection points) to each reflection point determined to be the observation target reflection point. The observation target detection device 1 then determines whether or not the total number Q of reflection points determined to be the non-observation target reflection points q is equal to or more than 2 (Q≥2, Step S051). When Q≥2 (Step S051; Yes), the observation target detection device 1 then executes displacement calculation processing (Step S006, sixth step) to calculate the displacement at each reflection point determined as the observation target reflection point by the second determination unit 15 and, as a non-observation target reflection point displacement, the displacement corresponding to the reflection point with the maximum signal amplitude among the reflection points determined as the non-observation target reflection points by the second determination unit 15. FIG. 12 is a flowchart showing an example of the displacement calculation processing (sixth step S006).

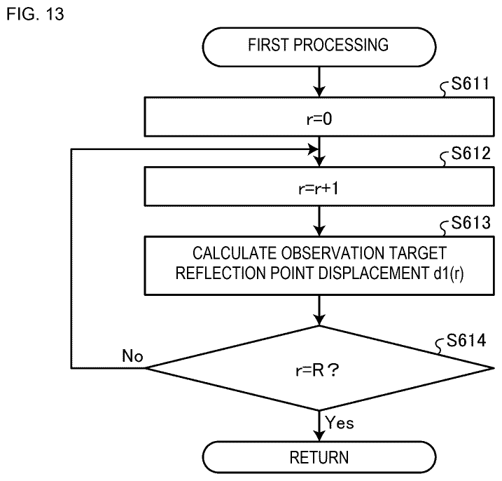

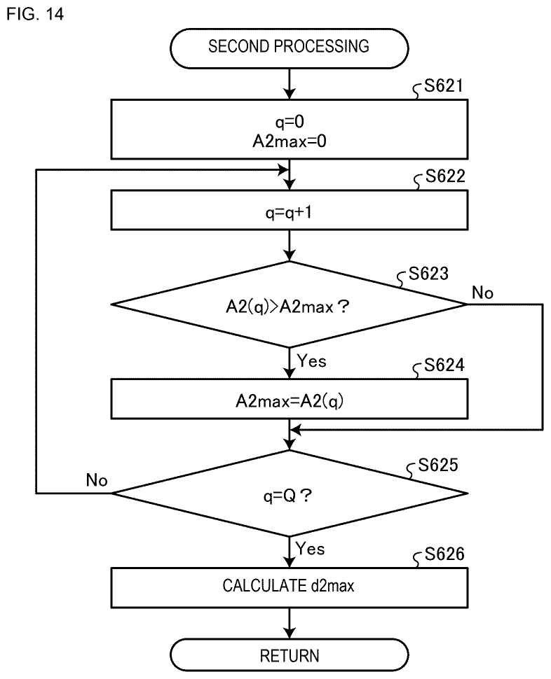

In the displacement calculation processing (sixth step S006), the displacement calculation unit 16 executes first processing (Step S610) to calculate the displacement at each reflection point determined as the observation target reflection point, and also executes second processing (Step S620) to calculate the displacement corresponding to the reflection point with the maximum signal amplitude among the reflection points determined as the non-observation target reflection points. FIG. 13 is a sub-flowchart showing an example of the first processing of the displacement calculation processing (sixth step S006). FIG. 14 is a sub-flowchart showing an example of the second processing of the displacement calculation processing (sixth step S006).

In the first processing of the displacement calculation processing (sixth step S006) shown in FIG. 13, the displacement calculation unit 16 first initializes the number r of the reflection point determined as the observation target reflection point by the second determination unit 15. Specifically, the displacement calculation unit 16 sets the number r to “0” (r=0, Step S611). The displacement calculation unit 16 then increments the number r (r=r+1, Step S612) and uses Formula (4) above to calculate the displacement at the observation target reflection point r (observation target reflection point displacement d1 (r)) (Step S613).

After calculating the observation target reflection point displacement d1(r) (Step S613), the displacement calculation unit 16 determines whether or not the number r is R (Step S614). When the number r is not R (Step S614; No), the processing is repeated from Step S612. When the number r is R (Step S614; Yes), the processing moves to the second processing of the displacement calculation processing (sixth step S006) shown in FIG. 14. Through the first processing of the displacement calculation processing (sixth step S006) described above, the displacements are calculated at all the reflection points determined as the observation target reflection points by the second determination unit 15.

Once the processing moves to the second processing of the displacement calculation processing (sixth step S006) shown in FIG. 14, the displacement calculation unit 16 first initializes the number q of the reflection point determined as the non-observation target reflection point by the second determination unit 15, and the maximum value A2max of the amplitude A2(q) at the non-observation target reflection point q among the non-observation target reflection points q numbered 1 to Q. Specifically, the displacement calculation unit 16 sets the number q and the maximum value A2max to “0” (q=0, A2max=0, Step S621).

Then, the displacement calculation unit 16 increments the number q (q=q+1, Step S622) and determines whether or not the amplitude A2 (q) of the signal at the non-observation target reflection point q exceeds the maximum value A2max (A2 (q)>A2max, Step S623). When the amplitude A2 (q) of the signal at the non-observation target reflection point q is equal to or smaller than the maximum value A2max (A2 (q)≤A2max, Step S623; No), the processing moves to Step S625. When the amplitude A2(q) of the signal at the non-observation target reflection point q exceeds the maximum value A2max (A2(q)>A2max, Step S623; Yes), the displacement calculation unit 16 updates the amplitude A2(q) of the signal at the non-observation target reflection point q to the maximum value A2max (A2max=A2(q), Step S624).

After updating the amplitude A2(q) of the signal at the non-observation target reflection point q to the maximum value A2max (Step S624), the displacement calculation unit 16 determines whether or not the number q is Q (Step S625). When the number q is not Q (Step S625; No), the processing is repeated from Step S622. When the number q is Q (Step S625; Yes), the displacement calculation unit 16 uses Formula (4) above to calculate the displacement d2max at the non-observation target reflection point q where the signal amplitude A2(q) has been updated to the maximum value A2max in Step S624 (Step S626). Then, the processing returns to the observation target detection processing shown in FIG. 7. Through the second processing of the displacement calculation processing (sixth step S006) described above, the displacement corresponding to the non-observation target reflection point with the maximum signal amplitude, among the reflection points determined as the non-observation target reflection points by the second determination unit 15, is extracted as the non-observation target reflection point displacement d2max.

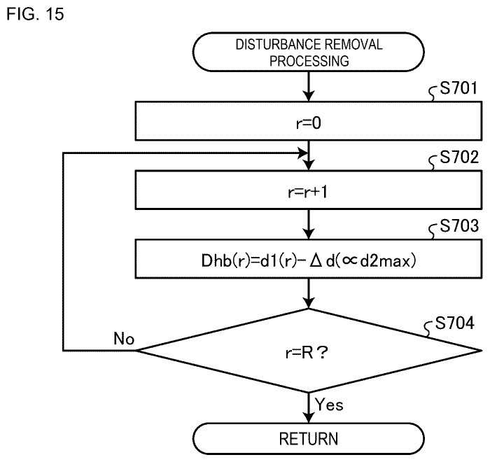

Back to the observation target detection processing shown in FIG. 7, the observation target detection device 1 then executes disturbance removal processing (Step S007, seventh step) to remove the displacement component of the stationary object from the displacement at the observation target reflection point calculated by the displacement calculation unit 16 to generate a body surface displacement of the human body. FIG. 15 is a flowchart showing an example of the disturbance removal processing (seventh step S007).

In the disturbance removal processing (seventh step S007), the disturbance removal unit 17 initializes the number r of the reflection point determined as the observation target reflection point by the second determination unit 15. Specifically, the disturbance removal unit 17 sets the number r to “0” (r=0, Step S701).

The disturbance removal unit 17 then increments the number r (r=r+1, Step S702) and removes the displacement component Δd proportional to the observation target reflection point displacement d2max extracted through the second processing of the displacement calculation processing (sixth step S006) from the observation target reflection point displacement d1(r) to generate the body surface displacement Dhb (r) of the human body (Step S703). Specifically, the disturbance removal unit 17 calculates the body surface displacement Dhb (r) of the human body by subtracting the displacement component Δd proportional to the observation target reflection point displacement d2max from the observation target reflection point displacement d1(r).

After removing the displacement component Δd proportional to the observation target reflection point displacement d2max from the observation target reflection point displacement d1(r) (Step S703), the disturbance removal unit 17 determines whether or not the number r is R (Step S704). When the number r is not R (Step S704; No), the processing is repeated from Step S702. When the number r is R (Step S704; Yes), the processing returns to the observation target detection processing shown in FIG. 7 and ends the observation target detection processing. Through the disturbance removal processing (seventh step S007) described above, the body surface displacement can be acquired, in which the displacement component caused by the periodic vibration of the observation target detection device 1 is suppressed at all reflection points determined as the human body that is the observation target of the observation target detection device 1 by the second determination unit 15.

Note that, when the total number N of the reflection points n selected in the first determination processing (third step S003) by the first determination unit 13 is 0 or 1, the subsequent second correlation degree calculation processing (Step S004, fourth step) by the second correlation degree calculation unit 14 cannot be executed. When the total number N of the reflection points n selected in the first determination processing (third step) by the first determination unit 13 is 2, only one second correlation degree cor2(n1, n2) is calculated in the subsequent second correlation degree calculation processing (Step S004, fourth step) by the second correlation degree calculation unit 14. In this case, when the two reflection points n1 and n2 used for the calculation of the second correlation degree cor2(n1, n2) are both determined as the observation target reflection points r in the further subsequent second determination processing (Step S005, fifth step) by the second determination unit 15, in other words, when the two reflection points n1 and n2 used for the calculation of the second correlation degree cor2(n1, n2) are not determined as the non-observation target reflection points q, this may include both a case where both of these two reflection points n1 and n2 are human bodies and a case where one is a human body and the other is a stationary object. Therefore, when the total number N of the reflection points n selected by the first determination processing (third step S003) is less than 3 (N<3, Step S031; No), in other words, when the total number N of the reflection points n selected by the first determination processing (third step S003) is equal to or less than 2 (N≤2), the processing subsequent to the second correlation degree calculation processing (Step S004, fourth step) is canceled, and the observation target detection processing is terminated.

In the second determination processing (fifth step S005) by the second determination unit 15, when the total number Q of the reflection points determined as the non-observation target reflection points q is 0, in other words, when all the reflection points n selected in the first determination processing (third step S003) by the first determination unit 13 are determined as the observation target reflection points r, this may include a case where one of the R reflection points determined as the observation target reflection points r is a stationary object. In this case, it is not possible to calculate the non-observation target reflection point displacement d2 (q) in the displacement calculation processing (sixth step S006) by the displacement calculation unit 16, and to extract the maximum value d2max of the non-observation target reflection point displacement in the disturbance removal processing (seventh step S007) by the disturbance removal unit 17. This makes it impossible to remove the displacement component proportional to the maximum value d2max of the non-observation target reflection point displacement from the observation target reflection point displacement d1(r). Therefore, when the total number Q of the reflection points determined as the non-observation target reflection points q is less than 2 (Q<2, Step S051; No), in other words, when all the reflection points n are determined as the observation target reflection points r (R=N), the processing subsequent to the displacement calculation processing (Step S006, sixth step) is canceled, and the observation target detection processing is terminated.

Through such observation target detection processing in the observation target detection device 1 and the observation target detection method according to the embodiment, the positions of reflection points within the observation range 2 are specified in the position specification processing (Step S001, first step) by the radar 11, and the first correlation degree calculation processing (Step S002, second step) is executed by the first correlation degree calculation unit 12 on each reflection point specified in the position specification processing to calculate a first correlation degree indicating the level of correlation between temporal changes in at least one first signal property, e.g., the amplitude, intensity, and power of the signal, at each reflection point and temporal changes in phase. Then, the first determination unit 13 executes the first determination processing (Step S003, third step) to select a reflection point at which the first correlation degree calculated in the first correlation degree calculation processing (Step S002, second step) by the first correlation degree calculation unit 12 is equal to or greater than a predetermined threshold. This allows selection of a reflection point including a periodic fluctuation component (a substantially periodic body surface displacement of the human body such as breathing and heartbeat, or periodic vibration of the observation target detection device 1) that is relatively large compared to random noise components.

The second correlation degree calculation unit 14 executes the second correlation degree calculation processing (Step S004, fourth step) to calculate a second correlation degree indicating the level of correlation between temporal changes in at least one first signal property, e.g., the amplitude, intensity, power, and phase between the signals at the reflection points selected in the first determination processing. Then, the second determination unit 15 executes the second determination processing (Step S005, fifth step) to determine that each of the reflection points at which the second correlation degree calculated in the second correlation degree calculation processing is equal to or greater than a predetermined threshold is the non-observation target reflection point, and that reflection points other than the non-observation target reflection point are the observation target reflection points. As a result, among the reflection points selected in the first determination processing, reflection points including no body surface displacement component of a human body are determined as the non-observation target reflection points, and reflection points including body surface displacement components of a human body are determined as the observation target reflection points.

Therefore, the observation target detection processing according to the embodiment makes it possible to appropriately discriminate between the observation target reflection points including the body surface displacement of the human body and the non-observation target reflection points that are stationary objects including no body surface displacement of the human body.

The displacement calculation unit 16 executes the displacement calculation processing (Step S006, sixth step) to calculate the displacement at each reflection point determined as the observation target reflection point by the second determination unit 15, and the displacement corresponding to the reflection point with the maximum signal amplitude, intensity, or power among the reflection points determined as the non-observation target reflection points by the second determination unit 15. Then, the disturbance removal unit 17 executes the disturbance removal processing (Step S007, seventh step) to remove the displacement component of the non-observation target reflection point determined to be a stationary object from the displacement at the observation target reflection point calculated by the displacement calculation unit 16. This makes it possible to acquire a highly accurate body surface displacement of the human body as biometric information by removing the displacement component caused by the periodic vibration of the observation target detection device 1 (radar 11) from the observation target reflection point displacement including the body surface displacement of the human body and the displacement component caused by the periodic vibration of the observation target detection device 1 (radar 11).

The embodiment described above is intended to facilitate understanding of the present disclosure, and not intended to limit the present invention. The present disclosure may be modified or improved without departing from the spirit thereof, and equivalents are also included in the present disclosure.

The present disclosure may adopt the following configurations as described above or instead of the above.

(1) An observation target detection device according to an aspect of the present disclosure includes: a radar that emits radio waves into an observation range to specify, based on reflected waves of the radio waves, a position of a reflection point within the observation range; a first correlation degree calculation unit that calculates a first correlation degree indicating a level of correlation between temporal changes in at least one of amplitude, intensity, and power of a signal at the reflection point and temporal changes in phase; a first determination unit that selects a reflection point at which the first correlation degree is equal to or greater than a predetermined value; a second correlation degree calculation unit that calculates, when a number of reflection points selected by the first determination unit is equal to or more than three, a second correlation degree indicating a level of correlation between temporal changes in at least one of the amplitude, intensity, power, and phase between signals at two of the reflection points; and a second determination unit that determines that one of the reflection points at which the second correlation degree is equal to or greater than a predetermined value is a non-observation target reflection point and that one of the reflection points other than the non-observation target reflection point is an observation target reflection point.

In this configuration, for each reflection point specified by the radar, a reflection point including a periodic fluctuation component that is relatively large compared to random noise components is selected by calculating the first correlation degree indicating the level of correlation between temporal changes in at least one of the amplitude, intensity, and power of the signal at each reflection point and temporal changes in phase and selecting a reflection point at which the calculated first correlation degree is equal to or greater than a predetermined threshold. Then, the second correlation degree indicating the level of correlation between temporal changes in at least one of the amplitude, intensity, power, and phase between the signals of the selected reflection points is calculated. Each reflection point at which the second correlation degree is equal to or greater than a predetermined threshold is determined as a non-observation target reflection point, and reflection points other than the non-observation target reflection point are determined as observation target reflection points. This makes it possible to appropriately discriminate between the observation target reflection point and the non-observation target reflection points including no displacement at the observation target reflection point.

(2) In the observation target detection device according to (1) above, the observation target detection device further includes: a displacement calculation unit that calculates a displacement at the observation target reflection point and a displacement at the non-observation target reflection point; and a disturbance removal unit that removes a displacement component at the non-observation target reflection point from the displacement at the observation target reflection point.

In this configuration, the displacement at each of the reflection points determined as the non-observation target reflection point and the observation target reflection point is calculated, and the displacement component of the non-observation target reflection point is removed from the displacement at the observation target reflection point. This makes it possible to acquire the displacement at the observation target reflection point with high accuracy by removing the displacement component caused by the periodic vibration of the observation target detection device from the observation target reflection point displacement, which includes the displacement at the observation target reflection point and the displacement component caused by the periodic vibration of the observation target detection device.

(3) In the observation target detection device according to (2) above, an object determined as the observation target reflection point by the second determination unit is a human body, and an object determined as the non-observation target reflection point by the second determination unit is a stationary object other than a human body within the observation range.

This configuration makes it possible to appropriately discriminate between the observation target reflection point including the body surface displacement components of the human body and the non-observation target reflection point that is a stationary object including no body surface displacement components of the human body.

(4) In the observation target detection device according to (3) above, the disturbance removal unit removes a displacement component of the stationary object from the displacement at the observation target reflection point to generate a body surface displacement of the human body.

This configuration makes it possible to acquire a highly accurate body surface displacement of the human body as biometric information by removing the displacement components caused by the periodic vibration of the observation target detection device from the observation target reflection point displacement, which includes the body surface displacement of the human body and the displacement components caused by the periodic vibration of the observation target detection device.

(5) An observation target detection method according to an aspect of the present disclosure includes: a first step of emitting radio waves into an observation range of a radar to specify, based on reflected waves of the radio waves, a position of a reflection point within the observation range; a second step of calculating a first correlation degree indicating a level of correlation between temporal changes in at least one of amplitude, intensity, and power of a signal at the reflection point and temporal changes in phase; a third step of selecting a reflection point at which the first correlation degree is equal to or greater than a predetermined value; a fourth step of calculating, when a number of reflection points selected in the third step is equal to or more than three, a second correlation degree indicating a level of correlation between temporal changes in at least one of the amplitude, intensity, power, and phase between signals at two of the reflection points; and a fifth step of determining that one of the reflection points at which the second correlation degree is equal to or greater than a predetermined value is a non-observation target reflection point and that one of the reflection points other than the non-observation target reflection point is an observation target reflection point.

According to this configuration, the position of the reflection point within the observation range 2 of the radar is specified in position specification processing (first step S001), and first correlation degree calculation processing (second step S002) is executed on each reflection point specified in the position specification processing to calculate the first correlation degree indicating the level of correlation between temporal changes in at least one of the amplitude, intensity, and power of the signal at each reflection point and temporal changes in phase. Then, first determination processing (third step S003) is executed to select a reflection point at which the first correlation degree is equal to or greater than a predetermined threshold, thereby selecting a reflection point including a periodic fluctuation component that is relatively large compared to a random noise component. Thereafter, second correlation degree calculation processing (fourth step S004) is executed to calculate a second correlation degree indicating the level of correlation between temporal changes in amplitude, intensity, power, and phase between the signals at the reflection points selected in the first determination processing. Then, second determination processing (fifth step S005) is executed to determine that one of the reflection points at which the second correlation degree calculated in the second correlation degree calculation processing is equal to or greater than a predetermined threshold is a non-observation target reflection point, and that one of the reflection points other than the non-observation target reflection point is an observation target reflection point. This makes it possible to appropriately discriminate between the observation target reflection point and the non-observation target reflection point including no displacement at the observation target reflection point.

(6) In the observation target detection method according to (5), the fourth step and the fifth step are canceled when the number of reflection points selected in the third step is equal to or less than two.

(7) In the observation target detection method according to (5), the observation target detection method further includes: a sixth step of calculating a displacement at the observation target reflection point and a displacement at the non-observation target reflection point; and a seventh step of removing a displacement component at the non-observation target reflection point from the displacement at the observation target reflection point.

According to this configuration, displacement calculation processing (sixth step S006) is executed to calculate the displacement at each of the reflection points determined as the non-observation target reflection point and the observation target reflection point, and disturbance removal processing (seventh step S007) is executed to remove the displacement component of the non-observation target reflection point from the displacement at the observation target reflection point. This makes it possible to acquire the displacement at the observation target reflection point with high accuracy by removing the displacement component caused by the periodic vibration of the radar from the observation target reflection point displacement, which includes the displacement at the observation target reflection point and the displacement component caused by the periodic vibration of the radar.

(8) In the observation target detection method according to (7), the sixth step and the seventh step are canceled when all reflection points are determined to be the observation target reflection point in the fifth step.

(9) In the observation target detection method according to (7), an object determined as the observation target reflection point in the fifth step is a human body, and an object determined as the non-observation target reflection point in the fifth step is a stationary object other than a human body within the observation range.

This configuration makes it possible to appropriately discriminate between the observation target reflection point including the body surface displacement components of the human body and the non-observation target reflection point that is a stationary object including no body surface displacement components of the human body.

(10) In the observation target detection method according to (9), in the seventh step, a displacement component of the stationary object is removed from the displacement at the observation target reflection point to generate a body surface displacement of the human body.

This configuration makes it possible to acquire a highly accurate body surface displacement of the human body as biometric information by removing the displacement components caused by the periodic vibration of the observation target detection device from the observation target reflection point displacement, which includes the body surface displacement of the human body and the displacement components caused by the periodic vibration of the observation target detection device.

The present disclosure makes it possible to realize an observation target detection device and an observation target detection method capable of appropriately discriminating between an observation target and a non-observation target.

### REFERENCE SIGNS LIST

1 OBSERVATION TARGET DETECTION DEVICE
2 OBSERVATION RANGE
11 RADAR
12 FIRST CORRELATION DEGREE CALCULATION UNIT
13 FIRST DETERMINATION UNIT
14 SECOND CORRELATION DEGREE CALCULATION UNIT
15 SECOND DETERMINATION UNIT
16 DISPLACEMENT CALCULATION UNIT
17 DISTURBANCE REMOVAL UNIT

1. An observation target detection device comprising:
a radar configured to emit radio waves to identify a plurality of reflection points within an observation range based on reflected radio waves; and
circuitry configured to:
calculate, for each of the plurality of reflection points, a first correlation degree indicating a correlation between temporal changes in a first signal property and temporal changes in phase of a signal from a respective reflection point;
select a set of candidate reflection points having the first correlation degree that is equal to or greater than a first predetermined value;
calculate, when a number of reflection points selected is equal to or more than three, for pairs of the candidate reflection points, a second correlation degree indicating a correlation between temporal changes in the first signal property between signals at each of the pair of the candidate reflection points; and
determine, for pairs of reflection points having the second correlation degree equal to or greater than a second predetermined value to be a non-observation target reflection points and otherwise determine the pair of reflection to be observation target reflection points.

2. The observation target detection device according to claim 1, wherein the circuitry is further configured to:
calculate a displacement at the observation target reflection point and a displacement at the non-observation target reflection point; and
remove a displacement component at the pair of non-observation target reflection points from the displacement at the pair of observation target reflection points.

3. The observation target detection device according to claim 2, wherein
an object determined as the observation target reflection point is a human body, and
an object determined as the non-observation target reflection point is a stationary object other than a human body within the observation range.

4. The observation target detection device according to claim 3, wherein the circuitry is configured to remove a displacement component of the stationary object from the displacement at the observation target reflection points to generate a body surface displacement of the human body.

5. The observation target detection device according to claim 1, wherein the first signal property is at least one of amplitude, intensity, and power of a signal.

6. The observation target detection device according to claim 1, wherein the circuitry is further configured to:
calculate a displacement of the observation target reflection point based on the phase of the signal from the observation target reflection point;
calculate a displacement of at least one non-observation target reflection point based on the phase of the signal from the at least one non-observation target reflection point; and
remove a displacement component corresponding to the displacement of the at least one non-observation target reflection point from the displacement of the observation target reflection point.

7. The observation target detection device according to claim 6, wherein to remove the displacement component the circuitry is further configures to:
identify, from among the one or more non-observation target reflection points, a reference non-observation target reflection point having a maximum signal amplitude; and
subtract a displacement component proportional to a displacement of the reference non-observation target reflection point from the displacement of the observation target reflection point to generate a body surface displacement.

8. The observation target detection device according to claim 1, wherein the first predetermined value is 0.9.

9. An observation target detection method comprising:
emitting radio waves to identify a plurality of reflection points within an observation range of a radar, based on reflected waves of the radio waves;
calculating, for each of the plurality of reflection points, a first correlation degree indicating correlation between temporal changes in a first signal property;
selecting a set of candidate reflection points, wherein each candidate reflection point has the first correlation degree equal to or greater than a first predetermined value;
calculating, when a number of reflection points selected is equal to or more than three, for pairs of the candidate reflection points, a second correlation degree indicating correlation between temporal changes in the first signal property between signals from each pair of candidate reflection points; and
determining, from the set of candidate reflection points having the second correlation degree equal to or greater than a second predetermined value to be non-observation target reflection points and otherwise determine that the pair of reflection points are observation target reflection points.

10. The observation target detection method according to claim 9, wherein
when the number of reflection points selected is equal to or less than two, ending the method.

11. The observation target detection method according to claim 9, further comprising:
calculating a displacement at the observation target reflection points and a displacement at the non-observation target reflection points; and
removing a displacement component at the non-observation target reflection points from the displacement at the observation target reflection points.

12. The observation target detection method according to claim 11, wherein
when all reflection points are determined to be the observation target reflection point not performing the calculating and removing the displacement.

13. The observation target detection method according to claim 11, wherein
an object determined as the observation target reflection point is a human body, and
an object determined as the non-observation target reflection point is a stationary object other than a human body within the observation range.

14. The observation target detection method according to claim 13, further comprising generating a body surface displacement of the human body by removing a displacement component of the stationary object from the displacement at the observation target reflection point.

15. The observation target detection method according to claim 9, wherein the first signal property is at least one of amplitude, intensity, and power of a signal.

16. A non-transitory computer-readable medium storing instructions that, when executed by a processor of an observation target detection device, cause the device to perform a method comprising:
receiving data corresponding to a plurality of reflection points within an observation range identified by a radar;
calculating, for each of the plurality of reflection points, a first correlation degree indicating a correlation between temporal changes in a first signal property selecting a set of candidate reflection points from the plurality of reflection points, wherein each candidate reflection point has a first correlation degree that is greater than or equal to a first predetermined value;
calculating, when the set of candidate reflection points includes at least three reflection points, a second correlation degree for pairs of the candidate reflection points, the second correlation degree indicating a correlation between signals from each pair of candidate reflection points; and
determining that a pair of candidate reflection points having a second correlation degree greater than or equal to a second predetermined value are non-observation target reflection points and otherwise are observation target reflection points.

17. The non-transitory computer-readable medium of claim 16, wherein the method further comprises:
calculating an observation target displacement based on a phase of a signal from the observation target reflection point;
calculating a non-observation target displacement based on a phase of a signal from a non-observation target reflection point; and
generating a body surface displacement value by removing a component related to the non-observation target displacement from the observation target displacement.

18. The non-transitory computer-readable medium of claim 16, wherein when the set of candidate reflection points includes fewer than three reflection points, stopping the method.

19. The non-transitory computer-readable medium of claim 16, wherein the first signal property is at least one of amplitude, intensity, and power of a signal.
